SEQUENCE LISTING (1 sequence)

SEQ ID NO: 1 - DNA, 19 bp, synthetic construct
  source 1..19: mol_type = other DNA
gcgtggtaccacgcttttt

(19) United States
(12) Patent Application Publication
(10) Pub. No.: US 2026/0224711 A1
(43) Pub. Date: Aug. 6, 2026

(54) ENHANCING BRAIN PERMEABILITY AND ACTIVITY OF GLUCOSYLCERAMIDASE (GCase) PROSNA FOR NEURODEGENERATIVE DISEASES

(71) Applicant: NORTHWESTERN UNIVERSITY, Evanston, IL (US)

(72) Inventors: Chad A. Mirkin, Wilmette, IL (US); Sergej Kudruk, Evanston, IL (US); Julianna Nicole Bourgeois, Evanston, IL (US); Max Everett Distler, Evanston, IL (US); Caroline Danielle Kusmierz, San Antonio, TX (US)

(21) Appl. No.: 19/149,326

(22) PCT Filed: Jan. 19, 2024
(86) PCT No.: PCT/US24/12138

§ 371 Date: Jul. 18, 2025

Related U.S. Application Data
(60) Provisional application No. 63/480,857, filed on Jan. 20, 2023.

(51) Int. Cl. A61K 47/54; A61K 38/47; A61K 47/55; C12N 9/24

(52) U.S. Cl. CPC A61K 47/549; A61K 38/47; A61K 47/551; C12N 9/2402; C12Y 302/01045

(57) ABSTRACT

The present disclosure provides protein-core spherical nucleic acids (ProSNAs) and strategies for delivering glucosyl-ceramidase (GCase) proteins into cells (e.g., brain cells). In some aspects the disclosure provides a protein-core spherical nucleic acid (ProSNA) comprising: (a) a protein core that comprises a glucosylceramidase (GCase); and (b) a shell of oligonucleotides attached to the protein core.

### CROSS-REFERENCE TO RELATED APPLICATIONS

This application claims the priority benefit under 35 U.S.C. § 119 (e) of U.S. Provisional Application No. 63/480,857, filed Jan. 20, 2023, which is incorporated herein by reference in its entirety.

### STATEMENT OF GOVERNMENT INTEREST

This invention was made with government support under grant number 5P50CA221747-04 awarded by the National Institutes of Health and grant number FA8650-15-2-5518 awarded by the Air Force Research Laboratory (AFRL). The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2022-186_SeqListing.XML”, which was created on Jan. 18, 2024 and is 1,968 bytes in size. The subject matter of the Sequence Listing is incorporated herein in its entirety by reference.

### BACKGROUND

The global burden of money spent on the treatment of neurodegenerative diseases and their symptoms is increasing every year. In 2017, the sums spent by the healthcare system in Europe and the USA amounted to an estimated 1.6 trillion dollars.

### SUMMARY

As the average age of the population continues to rise, the importance of treatments for neurodegenerative diseases continues to grow. The problems in the design of suitable therapeutics are very complex and must cover areas from efficient blood-brain barrier transport to successful restoration of enzyme activities in defined brain areas of diseased patients. A large proportion of Parkinson and Gaucher Type II and Ill patients have a mutation in the GBA1 gene which causes misfolding and subsequent deactivation of the glucosylceramidase (GCase) protein. To re-establish autophagy function in lysosomes, it is necessary to either reactivate the misfolded GCase protein or to specifically deliver the functional protein into the lysosomes of microglial cells. In various aspects, the present disclosure provides protein spherical nucleic acids (ProSNAs) and very powerful and elegant methods of using the ProSNAs to deliver targeted protein into the endolysosomal organelle of the cells. Because iron transport and vitamin transport are essential for metabolic exchanges between blood and brain areas, the abundancy of transferrin receptors and folate receptors in brain endothelial cells is greatly increased. The present disclosure therefore provides a ProSNA GCase design that harbors a transferrin aptamer and folic acid (vitamin B9) in the terminal DNA position. Using this multivalent GCase design, a significant increase in the rate of transcytosis across the blood-brain barrier was observed while maintaining its increased function.

Applications of the technology disclosed herein include but are not limited to:
Enzyme Replacement Therapy for Neurological Diseases
Blood Brain Barrier Transport
Enzyme Activation/Maintenance of Enzyme Activity

Advantages of the technology disclosed herein include but are not limited to:
Increased Transcytosis across the Blood Brain Barrier
Increased Enzyme Activity
Elevated Enzyme Stability.
No Liposomal Transport Carriers required
Straight forward Synthesis

Thus, in some aspects the disclosure provides a GCase ProSNA-TfF ((T)rans (F)errin receptor and (F)olate receptor) comprising (i) a protein core comprising a GCase and (ii) one or more folate modified transferrin aptamers, which are radially arranged around the protein core. The GCase-TfF structure enables a blood brain barrier transport of an activated protein. In some embodiments, the GCase is a quetiapine, Saposin-C, or Ambroxol assembled GCase. In some embodiments, the GCase is a quetiapine or Ambroxol assembled GCase.

In some aspects, the disclosure provides a protein-core spherical nucleic acid (ProSNA) comprising: (a) a protein core that comprises a glucosylceramidase (GCase); and (b) a shell of oligonucleotides attached to the protein core. In some embodiments, the GCase is an activated GCase. In further embodiments, the GCase is a non-activated GCase. In some embodiments, one or more oligonucleotides in the shell of oligonucleotides is a bivalent ligand. In some embodiments, one or more oligonucleotides in the shell of oligonucleotides comprises a bivalent ligand. In various embodiments, one or more oligonucleotides in the shell of oligonucleotides is a folate modified transferrin aptamer. In any of the aspects or embodiments, of the disclosure, each oligonucleotide in the shell of oligonucleotides is a folate modified transferrin aptamer. In some embodiments, the folate-modified transferrin aptamer comprises folic acid in the terminal position of the folate-modified transferrin aptamer. In various embodiments, the shell of oligonucleotides comprises one or more additional oligonucleotides. In some embodiments, one or more oligonucleotides in the shell of oligonucleotides is modified on its 5′ end and/or 3′ end with dibenzocyclooctyl (DBCO). In various embodiments, the shell of oligonucleotides comprises single-stranded DNA, double-stranded DNA, single-stranded RNA, double-stranded RNA, or a combination thereof. In some embodiments, each oligonucleotide in the shell of oligonucleotides is covalently attached to the protein core. In some embodiments, each oligonucleotide in the shell of oligonucleotides is attached to the protein core through a linker. In various embodiments, the linker is a cleavable linker or a non-cleavable linker. In some embodiments, the linker is SPDP succinimidyl 3-(2-pyridyldithio) propionate. In some embodiments, at least one oligonucleotide in the shell of oligonucleotides is attached to the protein core in the following configuration: protein core—NH—C(O)—(CH₂)₅—NH—C(O)—C₂—S—S-oligonucleotide-NH-PEG₃-Folate. In some embodiments, each oligonucleotide in the shell of oligonucleotides is attached to the protein core in the following configuration: protein core—NH—C(O)—(CH₂)₅—NH—C(O)—C₂—S—S-oligonucleotide-NH-PEG₃-Folate. In some embodiments, the linker is NHS-PEG_{(X)}-Azide, wherein X is 3 or 4. In some embodiments, at least one oligonucleotide in the shell of oligonucleotides is attached to the protein core in the following configuration: protein core—NH—(CO)-PEG_{X}-Triazole-oligonucleotide-NH—C(O)-PEG₃-Folate. In some embodiments, each oligonucleotide in the shell of oligonucleotides is attached to the protein core in the following configuration: protein core—NH—(CO)-PEG_{X}-Triazole-oligonucleotide-NH—C(O)-PEG₃-Folate. In some embodiments, one or more or all oligonucleotides in the shell of oligonucleotides is non-covalently attached to the protein core.

In some aspects, the disclosure provides a composition comprising a plurality of protein-core spherical nucleic acids (ProSNAs) of the disclosure.

In further aspects, the disclosure provides a method of treating, ameliorating, and/or preventing a synucleinopathy in a subject comprising administering to the subject an effective amount of (i) a ProSNA or composition of the disclosure. In some embodiments, the synucleinopathy is Lewy Body Dementia, Parkinson's disease, Gaucher's disease, or a combination thereof. In further embodiments, the Gaucher's disease is Type II or Type III.

In some aspects, the disclosure provides a method of delivering a glucosylceramidase (GCase) to a cell comprising contacting the cell with a ProSNA of the disclosure.

In further aspects, the disclosure provides a method of delivering a glucosylceramidase (GCase) to a cell comprising contacting the cell with a composition of the disclosure.

### BRIEF DESCRIPTION OF THE DRAWINGS

FIG. 1 depicts an exemplary synthesis of GCase ProSNA TfF.

FIG. 2 depicts a synthetic scheme for bivalent Transferrin-Folic Acid Ligand.

FIG. 3 shows MALDI-TOF spectra of successful transferrin aptamer synthesis.

FIG. 4 shows MALDI-TOF MS of successful bivalent ligand formation.

FIG. 5 shows Western Blot analysis of GCase with and without 10% betamercaptoethanol.

FIG. 6 depicts an exemplary schematic representation of bivalent ProSNA synthesis.

FIG. 7 shows SDS-PAGE analysis of unmodified protein (lane 1) and bivalent ProSNA (lane 3).

FIG. 8 shows GCase ProSNA Characterization. (a) UV-ViS analysis comparing the transferrin aptamer, folic acid-NHS, native protein, and bivalent GCase ProSNA. (b) Circular dichroism spectra of the native protein and the bivalent GCase ProSNA.

FIG. 9 shows results from a fluorometric GCase activity assay kit, using a synthetic substrate-4-Methylumbelliferyl β-D-glucopyranoside. Upon interaction with active GCase, this substrate releases a fluorophore detectable using a plate reader at Ex/Em=360/445 nm.

FIG. 10 shows results of a fluorometric GCase activity assay post-activation of native GCase with quetiapine at concentrations of either 0 μM, 5 μM, or 10 μM.

### DETAILED DESCRIPTION

Due to the continuously increasing average age of humans, the need for therapeutics for neurodegenerative diseases is increasing. A major problem in Parkinson's or Gaucher's disease is the irreversible aggregation of alpha synuclein into harmful Lewy bodies, which is partly driven by the abnormal accumulation of glucosylceramide lipid. The origin of the problem is the mutation of the GBA1 gene, which causes the misfolding of GCase, an enzyme responsible for the degradation of excess glucosylceramide. Targeting the blood-brain barrier (BBB) for drug delivery is a critical area of research, and the use of specific ligands via receptor-mediated transcytosis is a promising strategy. Folate and transferrin have been studied as potential ligands for facilitating transcytosis across the BBB. Folate, via the folate receptor, and transferrin, via the transferrin receptor (TfR), have been investigated for their potential to enhance the penetration of nanoparticles across the BBB. The use of these ligands, either individually or in combination, has shown promise for enhancing the penetration of drug delivery systems across the BBB. Using industry-established pluripotent stem cell culture systems, the technology disclosed herein helps not only to overcome the problem of selective blood-brain barrier transport, but also to successfully transport an activated and functional GCase enzyme into brain areas and restore the degradation of glucosylceramides.

### Terminology

All language such as “from,” “to,” “up to,” “at least,” “greater than,” “less than,” and the like include the number recited and refer to ranges which can subsequently be broken down into sub-ranges.

A range includes each individual member. Thus, for example, a group having 1-3 members refers to groups having 1, 2, or 3 members. Similarly, a group having 6 members refers to groups having 1, 2, 3, 4, or 6 members, and so forth.

As used in this specification and the appended claims, the articles “a” and “an” refer to one or to more than one (for example, to at least one) of the grammatical object of the article.

“About” and “approximately” shall generally mean an acceptable degree of error for the quantity measured given the nature or precision of the measurements. Exemplary degrees of error are within 20-25 percent (%), for example, within 20 percent, 10 percent, 5 percent, 4 percent, 3 percent, 2 percent, or 1 percent of the stated value or range of values.

The terms “polynucleotide” and “oligonucleotide” are interchangeable as used herein.

A “linker” as used herein is a moiety that joins an oligonucleotide to a protein core of a protein-core spherical nucleic acid (ProSNA), as described herein. In any of the aspects or embodiments of the disclosure, a linker is a cleavable linker, a non-cleavable linker, or a combination thereof.

A “subject” is a vertebrate organism. The subject can be a non-human mammal (e.g., a mouse, a rat, or a non-human primate), or the subject can be a human subject.

The terms “administering”, “administer”, “administration”, and the like, as used herein, refer to any mode of transferring, delivering, introducing, or transporting a therapeutic agent to a subject in need of treatment with such an agent. Such modes include, but are not limited to, intravenous, intraarterial, intraperitoneal, intranasal, intrathecal, and subcutaneous administration.

As used herein, “treating” and “treatment” refers to any reduction in the severity and/or onset of symptoms associated with a disorder as disclosed herein (e.g., a synucleinopathy). Accordingly, “treating” and “treatment” includes therapeutic and prophylactic measures. One of ordinary skill in the art will appreciate that any degree of protection from, or amelioration of, a disorder (e.g., a synucleinopathy) is beneficial to a subject, such as a human patient. The quality of life of a patient is improved by reducing to any degree the severity of symptoms in a subject and/or delaying the appearance of symptoms.

As used herein, a “monovalent” ligand is a single molecule that binds to a receptor or protein, while a “bivalent” ligand is a single molecule containing two discrete ligands connected by a spacer. Bivalent ligands can bind to two identical or different sites on the same target or on two different targets that are in close proximity. This can lead to increased potency or efficacy compared to monovalent ligands.

As used herein, a “multivalent” ligand is a ligand that can bind to two or more identical or different sites on the same target or on two or more different targets that are in close proximity.

As used herein, “activated” glucocerebrosidase (GCase) refers to the functional form of the enzyme, which has undergone a process of activation leading to its ability to catalyze the hydrolysis of substrates such as glucosylceramide. This activation involves coincubation with activators like quetiapine or ambroxol. Any activator known to activate GCase is contemplated by the disclosure. By contrast, “unactivated” GCase remains in an inactive state and is unable to effectively catalyze the hydrolysis of its substrates. The introduction of small molecule activators, such as ambroxol or quetiapine, aims to enhance the activity of GCase, particularly in the context of treating conditions as disclosed herein (for example and without limitation, Gaucher disease and GBA1-associated Parkinson's disease).

All references, patents, and patent applications disclosed herein are incorporated by reference with respect to the subject matter for which each is cited, which in some cases may encompass the entirety of the document.

### Protein-Core Spherical Nucleic Acids (ProSNAs)

Protein-core spherical nucleic acids (ProSNAs), which comprise a shell of oligonucleotides attached (e.g., covalently attached) to a protein core, have emerged as exciting new architectures with diverse biological applications in protein delivery, assembly, and intracellular detection [Brodin, J. D.; Sprangers, A. J.; McMillan, J. R.; Mirkin, C. A. DNA-Mediated Cellular Delivery of Functional Enzymes. J. Am. Chem. Soc. 2015, 137 (47), 14838-14841; Kusmierz, C. D.; Bujold, K. E.; Callmann, C. E.; Mirkin, C. A. Defining the Design Parameters for in Vivo Enzyme Delivery Through Protein Spherical Nucleic Acids. ACS Cent. Sci. 2020, 6 (5), 815-822]. The shell of oligonucleotides promotes cellular uptake, physiological stability and biocompatibility of protein relative to their individual components [Giljohann, D. A.; Seferos, D. S.; Patel, P. C.; Millstone, J. E.; Rosi, N. L.; Mirkin, C. A. Oligonucleotide Loading Determines Cellular Uptake of DNA-Modified Gold Nanoparticles. Nano Lett. 2007, 7 (12), 3818-3821].

A “protein-core” as used herein comprises glucosylceramidase (GCase). In any of the aspects or embodiments of the disclosure, the functionalized GCase is an activated GCase. In various embodiments, the functionalized GCase is a non-activated GCase. A GCase can be activated via coincubation with, for example and without limitation, quetiapine, or Ambroxol, or a combination thereof. Thus, in any of the aspects or embodiments of the disclosure, a GCase of the disclosure generally functions as the “core” of the protein-core SNA (SNA). A protein is a molecule comprising one or more polymers of amino acids. In various embodiments of the disclosure, a protein-core comprises or consists of a single protein (i.e., a single polymer of amino acids), a multimeric protein, a peptide (e.g., a polymer of amino acids that between about 2 and 50 amino acids in length), or a synthetic fusion protein of two or more proteins. Synthetic fusion proteins include, without limitation, an expressed fusion protein (expressed from a single gene) and post-expression fusions where proteins are conjugated together chemically. In any of the aspects or embodiments of the disclosure, a protein-core comprises or consists of a GCase. In any of the aspects or embodiments of the disclosure, a protein-core comprises or consists of Saposin C. In some embodiments, a ProSNA of the disclosure does not comprise polyethylene glycol (PEG). Proteins are understood in the art and may be either naturally occurring or non-naturally occurring.

Protein-Core SNA Synthesis. The disclosure provides compositions and methods in which one or more oligonucleotides (e.g., a folate modified transferrin aptamer) is associated with and/or attached to the surface of a protein-core SNA via a linker. The linker can be, in various embodiments, a cleavable linker, a non-cleavable linker, or a combination thereof. In some embodiments, a cleavable linker is sensitive to (and is cleaved in response to) a reducing agent (e.g., glutathione (GSH), dithiothreitol (DTT)) or a reducing environment (e.g., inside a cell). In various embodiments, a cleavable linker is sensitive to (and is cleaved in response to) various chemical stimuli such as, for example, acidity (e.g., low pH), an enzyme (e.g., peptidase), light (e.g., NIR laser), and/or hydrolysis.

Thus, the disclosure also provides methods of synthesizing a ProSNA as described herein. In general, a representative procedure for synthesizing protein-core SNAs (ProSNAs) includes attaching a desired amount of oligonucleotide to the surface of the protein. Attachment is performed by iterating over a two-step process: (1) attachment of linker to the surface of the protein and purification; (2) attachment of oligonucleotide (e.g., DNA) to the protein-conjugated linkers and purification. These two steps are repeated until a desired amount of oligonucleotide is attached to the protein. It will be understood that the foregoing procedure is exemplary in nature. In some embodiments, the methods generally comprise functionalization of GCase with one or more folate modified transferrin aptamer oligonucleotides. In some embodiments, the GCase is coincubated with, for example and without limitation, quetiapine, Ambroxol, or a combination thereof, thereby producing activated GCase. A crosslinker is added to the GCase (e.g., activated GCase or non-activated GCase) and then one or more folate terminated transferrin aptamer oligonucleotides are mixed with the GCase-linker, thereby producing the ProSNA. See also Example 1 and Example 2, below.

The linker links the protein-core to the oligonucleotide in the disclosed protein-core SNA (i.e., protein-core-LINKER-Oligonucleotide). In various embodiments, a single oligonucleotide is attached to a linker. In further embodiments, more than one oligonucleotide (e.g., two, three, or more) is attached to a single linker. In general, linkers contemplated by the disclosure include the following, which may be used solely or in combination in the ProSNAs of the disclosure: amide, thioether, triazole, oxime, urea, and thiourea. Additional linkers and attachment syntheses of the disclosure include those described in International Patent Application Publication No. WO 2022/183043, incorporated by reference herein in its entirety.

In some embodiments, the linker is SPDP succinimidyl 3-(2-pyridyldithio) propionate. In further embodiments, at least one oligonucleotide in the shell of oligonucleotides is attached to the protein core in the following configuration: protein core—NH—C(O)—(CH₂)₅—NH—C(O)—C₂—S—S-oligonucleotide-NH-PEG₃-Folate. In some embodiments, each oligonucleotide in the shell of oligonucleotides is attached to the protein core in the following configuration: protein core—NH—C(O)—(CH₂)₅—NH—C(O)—C₂—S—S-oligonucleotide-NH-PEG₃-Folate. In some embodiments, the linker is NHS-PEG_{(X)}-Azide, wherein X is 3 or 4. In still further embodiments, at least one oligonucleotide in the shell of oligonucleotides is attached to the protein core in the following configuration: protein core—NH—(CO)-PEG_{X}-Triazole-oligonucleotide-NH—C(O)-PEG₃-Folate. In some embodiments, each oligonucleotide in the shell of oligonucleotides is attached to the protein core in the following configuration: protein core—NH—(CO)-PEG_{X}-Triazole-oligonucleotide-NH—C(O)-PEG₃-Folate.

An oligonucleotide of the disclosure may be modified at either the 5′ terminus or the 3′ terminus for attachment to a protein core. In some embodiments, one or more oligonucleotides in the shell of oligonucleotides is modified on its 5′ end and/or 3′ end with dibenzocyclooctyl (DBCO).

An oligonucleotide of the disclosure can be modified at a terminus with an alkyne moiety, e.g., a DBCO-type moiety for reaction with the azide of the protein surface:

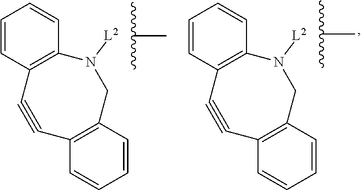

where L is a linker to a terminus of the polynucleotide. L² can be C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; wherein each Y is independently selected from the group consisting of a bond, C(O), O, NH, C(O)NH, and NHC(O); and m is 0, 1, 2, 3, 4, or 5. For example, the DBCO functional group can be attached via a linker having a structure of

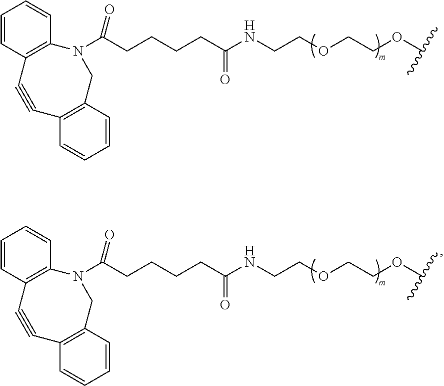

where the terminal “O” is from a terminal nucleotide on the polynucleotide. Use of this DBCO-type moiety results in a structure between the polynucleotide and the protein, in cases where a surface amine is modified, of:

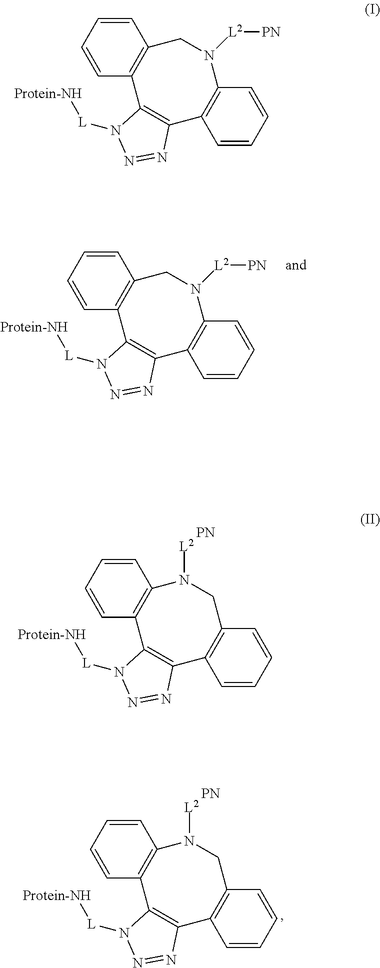

where L and L² are each independently selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and PN is the polynucleotide. Similar structures where a surface thiol or surface carboxylate of the protein are modified can be made in a similar fashion to result in comparable linkage structures.

The protein can be modified at a surface functional group (e.g., a surface amine, a surface carboxylate, a surface thiol) with a linker that terminates with an azide functional group: Protein-X-L-N₃, X is from a surface amino group (e.g., —NH—), carboxylic group (e.g., —C(O)— or —C(O)O—), or thiol group (e.g., —S—) on the protein; L is selected from C₁₋₁₀ alkylene, —Y—C(O)—C₁₋₁₀ alkylene-Y—, and —Y—C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); and m is 0, 1, 2, 3, 4, or 5. Introduction of the “L-N₃” functional group to the surface moiety of the protein can be accomplished using well-known techniques. For example, a surface amine of the protein can be reacted with an activated ester of a linker having a terminal N₃ to form an amide bond between the amine of the protein and the carboxylate of the activated ester of the linker reagent.

The oligonucleotide can be modified to include an alkyne functional group at a terminus of the oligonucleotide: Oligonucleotide-L₂-X—≡—R; L² is selected from C₁₋₁₀ alkylene, —C(O)—C₁₋₁₀ alkylene-Y—, and —C(O)—C₁₋₁₀ alkylene-Y—C₁₋₁₀ alkylene-(OCH₂CH₂)ₘ—Y—; each Y is independently selected from the group consisting of a bond, C(O), O, NH, C(O)NH, and NHC(O); m is 0, 1, 2, 3, 4, or 5; and X is a bond and R is H or C₁₋₁₀alkyl; or X and R together with the carbons to which they are attached form a 8-10 membered carbocyclic or 8-10 membered heterocyclic group. In some cases, the polynucleotide has a structure

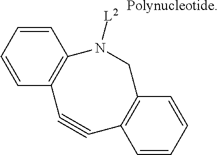

The protein, with the surface modified azide, and the polynucleotide, with a terminus modified to include an alkyne, can be reacted together to form a triazole ring in the presence of a copper (II) salt and a reducing agent to generate a copper (I) salt in situ. In some cases, a copper (I) salt is directly added. Contemplated reducing agents include ascorbic acid, an ascorbate salt, sodium borohydride, 2-mercaptoethanol, dithiothreitol (DTT), hydrazine, lithium aluminum hydride, diisobutylaluminum hydride, oxalic acid, Lindlar catalyst, a sulfite compound, a stannous compound, a ferrous compound, sodium amalgam, tris(2-carboxyethyl) phosphine, hydroquinone, and mixtures thereof.

The surface functional group of the protein can be attached to the oligonucleotide using other attachment chemistries. For example, a surface amine can be directly conjugated to a carboxylate or activated ester at a terminus of the oligonucleotide, to form an amide bond. A surface carboxylate can be conjugated to an amine on a terminus of the oligonucleotide to form an amide bond. Alternatively, the surface carboxylate can be reacted with a diamine to form an amide bond at the surface carboxylate and an amine at the other terminus. This terminal amine can then be modified in a manner similar to that for a surface amine of the protein. A surface thiol can be conjugated with a thiol moiety on the polynucleotide to form a disulfide bond. Alternatively, the thiol can be conjugated with an activated ester on a terminus of a polynucleotide to form a thiocarboxylate. Alternatively, the thiol can be conjugated with a Michael acceptor (e.g., a succinimide) on a terminus of a polynucleotide to form a thioether.

### Oligonucleotides

The disclosure provides protein-core spherical nucleic acids (ProSNAs) comprising a protein core and a shell of oligonucleotides attached to the exterior of the protein core. In any of the aspects or embodiments of the disclosure one or more or all of the oligonucleotides in the shell of oligonucleotides is an aptamer (e.g., a folate modified transferrin aptamer). An aptamer is a single-stranded oligonucleotide or peptide that fold into defined architectures and bind to targets such as proteins. Accordingly, all features and aspects of oligonucleotides described herein (e.g., length, type (DNA, RNA, modified forms thereof), optional presence of spacer) also apply to aptamers. Thus, the shell of oligonucleotides comprises, in various embodiments, one or more folate modified transferrin aptamer oligonucleotides. In further embodiments, the shell of oligonucleotides comprises a plurality of folate modified transferrin aptamer oligonucleotides. In any of the aspects or embodiments of the disclosure, each oligonucleotide in the shell of oligonucleotides is a folate modified transferrin aptamer. In any of the aspects or embodiments of the disclosure, the folate-modified transferrin aptamer comprises folic acid in the terminal 5′ or 3′ position of the folate-modified transferrin aptamer.

Oligonucleotides of the disclosure include, in various embodiments, DNA oligonucleotides, RNA oligonucleotides, modified forms thereof, or a combination thereof. In any aspects or embodiments described herein, an oligonucleotide is single-stranded, double-stranded, or partially double-stranded.

As described herein, modified forms of oligonucleotides are also contemplated by the disclosure which include those having at least one modified internucleotide linkage. In some embodiments, the oligonucleotide is all or in part a peptide nucleic acid. Other modified internucleoside linkages include at least one phosphorothioate or phosphodiester linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function, e.g., as a probe in hybridization. Examples of universal bases include but are not limited to 5′-nitroindole-2′-deoxyriboside, 3-nitropyrrole, inosine and hypoxanthine.

The term “nucleotide” or its plural as used herein is interchangeable with modified forms as discussed herein and otherwise known in the art. The term “nucleobase” or its plural as used herein is interchangeable with modified forms as discussed herein and otherwise known in the art. Nucleotides or nucleobases comprise the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30:613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, oligonucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide”.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

In still further embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂-described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligonucleotide consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}_C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}—, —CO—NR^{H}—CH₂—, —CH₂—NR^{H}—O—, —CH₂—NR^{H}—CO—, —O—NR^{H}—CH₂—, —O—NR^{H}, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—O—, —O—P(O,S)—O—, —O—P(S)₂—O—, —S—P(O)₂—O—, —S—P(O,S)—O—, —SP(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H} H—, —NR^{H}—P(O)₂—O—, —O—P(O,NR^{H})—O—, —CH₂—P(O)₂—O—, —O—P(O)₂—CH₂—, and —O—Si(R″)₂—O—; among which-CH₂—CO—NR^{H}—, —CH₂—NR^{H}—O—, —S—CH₂—O—, —O—P(O)₂—O—O—P(—O,S)—O—, —O—P(S)₂—O—, —NR^{H}P(O)₂—O—, —O—P(O,NR″)—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application No. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, or an RNA cleaving group. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Still other modifications include 2′-methoxy (2′-O—CH₃), 2′-aminopropoxy (2′-OCH₂CH₂CH₂NH₂), 2′-allyl(2′-CH₂—CH═CH₂), 2′-O-allyl(2′-O—CH₂—CH—CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In some aspects, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine (1H-pyrimido[5,4-b][1,4]benzoxazin-2 (3H)-one), phenothiazine cytidine (1H-pyrimido[5,4-b][1,4]benzothiazin-2 (3H)-one), G-clamps such as a substituted phenoxazine cytidine (e.g. 9-(2-aminoethoxy)-H-pyrimido[5,4-b][1,4]benzox-azin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30:613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various aspects, an oligonucleotide of the disclosure (e.g., a folate modified transferrin aptamer), or a modified form thereof, is generally about 5 nucleotides to about 100 nucleotides in length. More specifically, an oligonucleotide of the disclosure is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, about 10 to about 100 nucleotides in length, about 10 to about 90 nucleotides in length, about 10 to about 80 nucleotides in length, about 10 to about 70 nucleotides in length, about 10 to about 60 nucleotides in length, about 10 to about 50 nucleotides in length about 10 to about 45 nucleotides in length, about 10 to about 40 nucleotides in length, about 10 to about 35 nucleotides in length, about 10 to about 30 nucleotides in length, about 10 to about 25 nucleotides in length, about 10 to about 20 nucleotides in length, about 10 to about 15 nucleotides in length, about 18 to about 28 nucleotides in length, about 15 to about 26 nucleotides in length, about 15 to about 27 nucleotides in length, about 15 to about 28 nucleotides in length, about 15 to about 29 nucleotides in length, about 15 to about 30 nucleotides in length, and all oligonucleotides intermediate in length of the sizes specifically disclosed to the extent that the oligonucleotide is able to achieve the desired result. In further embodiments, an oligonucleotide of the disclosure is about 5 to about 100 nucleotides in length, about 5 to about 90 nucleotides in length, about 5 to about 80 nucleotides in length, about 5 to about 70 nucleotides in length, about 5 to about 60 nucleotides in length, about 5 to about 50 nucleotides in length, about 5 to about 40 nucleotides in length, about 5 to about 30 nucleotides in length, about 5 to about 20 nucleotides in length, about 5 to about 10 nucleotides in length, and all oligonucleotides intermediate in length of the sizes specifically disclosed to the extent that the oligonucleotide is able to achieve the desired result. Accordingly, in various embodiments, an oligonucleotide of the disclosure is or is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In further embodiments, an oligonucleotide of the disclosure is less than 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In some embodiments, an oligonucleotide of the disclosure is or is about 25 nucleotides in length. In various embodiments, the shell of oligonucleotides attached to the exterior of the protein core of the ProSNA comprises a plurality of oligonucleotides that all have the same length/sequence, while in some embodiments, the plurality of oligonucleotides comprises one or more oligonucleotides that have a different length and/or sequence relative to at least one other oligonucleotide in the plurality.

Methods of attaching detectable markers (e.g., fluorophores, radiolabels) and therapeutic agents (e.g., an antibody) as described herein to an oligonucleotide are known in the art.

Spacers. In some aspects and embodiments, one or more oligonucleotides in the shell of oligonucleotides that is attached to the protein core of a ProSNA comprise a spacer. “Spacer” as used herein means a moiety that serves to increase distance between the protein core and the oligonucleotide, or to increase distance between individual oligonucleotides when attached to the protein core in multiple copies, or to improve the synthesis of the ProSNA. Thus, spacers are contemplated being located between an oligonucleotide and the protein core.

In some aspects, the spacer when present is an organic moiety. In some aspects, the spacer is a polymer, including but not limited to a water-soluble polymer, a nucleic acid, a polypeptide, an oligosaccharide, a carbohydrate, a lipid, an ethylglycol, or a combination thereof. In any of the aspects or embodiments of the disclosure, the spacer is an oligo (ethylene glycol)-based spacer. In various embodiments, an oligonucleotide comprises 1, 2, 3, 4, 5, or more spacer (e.g., Spacer-18 (hexaethyleneglycol)) moieties. In further embodiments, the spacer is an alkane-based spacer (e.g., C12). In some embodiments, the spacer is an oligonucleotide spacer (e.g., T5). An oligonucleotide spacer may have any sequence that does not interfere with the ability of the oligonucleotides to become bound to the nanoparticle core or to a target. In certain aspects, the bases of the oligonucleotide spacer are all adenylic acids, all thymidylic acids, all cytidylic acids, all guanylic acids, all uridylic acids, or all some other modified base.

In various embodiments, the length of the spacer is or is equivalent to at least about 2 nucleotides, at least about 3 nucleotides, at least about 4 nucleotides, at least about 5 nucleotides, 5-10 nucleotides, 10 nucleotides, 10-30 nucleotides, or even greater than 30 nucleotides.

SNA surface density. Generally, a surface density of oligonucleotides that is at least about 2 pmoles/cm² will be adequate to provide a stable SNA. In some aspects, the surface density of a ProSNA of the disclosure is at least 5 pmoles/cm². Methods are also provided wherein the oligonucleotide is attached to the protein core of the ProSNA at a surface density of about 2 pmol/cm² to about 50 pmol/cm², about 2 pmol/cm² to about 45 pmol/cm², about 2 pmol/cm² to about 40 pmol/cm², about 2 pmol/cm² to about 35 pmol/cm², about 2 pmol/cm² to about 30 pmol/cm², about 2 pmol/cm² to about 25 pmol/cm², about 2 pmol/cm² to about 20 pmol/cm², about 2 pmol/cm² to about 15 pmol/cm², about 2 pmol/cm² to about 10 pmol/cm², about 2 pmol/cm² to about 5 pmol/cm², about 5 pmol/cm² to about 15 pmol/cm², or about 8 pmol/cm² to about 10 pmol/cm², or about 8 pmol/cm² to about 10.5 pmol/cm², or about 10 pmol/cm² to about 20 pmol/cm². In further embodiments, the surface density is, is about, or is at least about 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 19 pmol/cm², at least about 20 pmol/cm², at least about 25 pmol/cm², at least about 30 pmol/cm², at least about 35 pmol/cm², at least about 40 pmol/cm², at least about 45 pmol/cm², or at least about 50 pmol/cm² In further embodiments, the surface density is less than about 2 pmol/cm², less than about 3 pmol/cm², less than about 4 pmol/cm², less than about 5 pmol/cm², less than about 6 pmol/cm², less than about 7 pmol/cm², less than about 8 pmol/cm², less than about 9 pmol/cm², less than about 10 pmol/cm², less than about 15 pmol/cm², less than about 19 pmol/cm², less than about 20 pmol/cm², less than about 25 pmol/cm², less than about 30 pmol/cm², less than about 35 pmol/cm², less than about 40 pmol/cm², less than about 45 pmol/cm², or less than about 50 pmol/cm².

Alternatively, the density of oligonucleotide attached to the ProSNA is measured by the number of oligonucleotides attached to the ProSNA. With respect to the surface density of oligonucleotides attached to a ProSNA of the disclosure, it is contemplated that a ProSNA as described herein comprises about 1 to about 50 oligonucleotides on its surface. In various embodiments, a SNA comprises about 1 to about 50, or about 1 to about 45, or about 1 to about 40, or about 1 to about 35, or about 1 to about 30, or about 1 to about 25, or about 1 to about 20, or about 1 to about 15, or about 1 to about 10, or about 1 to about 5, or about 10 to about 50, or about 10 to about 45, or about 10 to about 40, or about 10 to about 35, or 10 to about 30, or about 10 to about 25, or about 10 to about 20, or about 10 to about 15 oligonucleotides in the shell of oligonucleotides attached to the protein core. In some embodiments, a ProSNA comprises about or less than about 18 oligonucleotides in the shell of oligonucleotides attached to the protein core. In some embodiments, a ProSNA comprises about or less than about 14-18 oligonucleotides in the shell of oligonucleotides attached to the protein core. In further embodiments, a ProSNA comprises about or at least about 1, 2, 3, 4, 5, 6, 7, 8, 9, 10, 11, 12, 13, 14, 15, 16, 17, 18, 20, 25, 30, 35, 40, 45, or 50 oligonucleotides in the shell of oligonucleotides attached to the protein core. In various embodiments, a ProSNA comprises less than about 2, 3, 4, 5, 6, 7, 8, 9, 10, 11, 12, 13, 14, 15, 16, 17, 18, 20, 25, 30, 35, 40, 45, or 50 oligonucleotides in the shell of oligonucleotides attached to the protein core. In further embodiments, a ProSNA consists of 1, 2, 3, 4, 5, 6, 7, 8, 9, 10, 11, 12, 13, 14, 15, 16, 17 18, 20, 25, 30, 35, 40, 45, or 50 oligonucleotides in the shell of oligonucleotides attached to the protein core. In still further embodiments, the shell of oligonucleotides attached to the protein core of the ProSNA comprises 1, 2, 3, 4, 5, 6, 7, 8, 9, 10, 11, 12, 13, 14, 15, 16, 17, 18, 19, 20 or more oligonucleotides. In some embodiments, the shell of oligonucleotides attached to the protein core of the ProSNA consists of 1, 2, 3, 4, 5, 6, 7, 8, 9, 10, 11, 12, 13, 14, 15, 16, 17, 18, 19, or 20 oligonucleotides.

### Compositions

The disclosure also provides compositions that comprise a ProSNA of the disclosure, or a plurality thereof. In some embodiments, the composition further comprises a pharmaceutically acceptable carrier. The term “carrier” refers to a vehicle within which the SNA as described herein is administered to a subject. Any conventional media or agent that is compatible with the ProSNAs according to the disclosure can be used. The term carrier encompasses diluents, excipients, adjuvants and a combination thereof. Pharmaceutically acceptable carriers are well known in the art (see, e.g., Remington's Pharmaceutical Sciences by Martin, 1975, the entire disclosure of which is herein incorporated by reference).

Exemplary “diluents” include water for injection, saline solution, buffers such as Tris, acetates, citrates or phosphates, fixed oils, polyethylene glycols, glycerine, propylene glycol or other synthetic solvents. Exemplary “excipients” include but are not limited to stabilizers such as amino acids and amino acid derivatives, polyethylene glycols and polyethylene glycol derivatives, polyols, acids, amines, polysaccharides or polysaccharide derivatives, salts, and surfactants; and pH-adjusting agents.

### Uses of ProSNAs to Treat a Disorder

In some embodiments, a ProSNA of the disclosure is used to treat a disorder. Thus, in some aspects, the disclosure provides methods of treating a disorder comprising administering an effective amount of a ProSNA or composition of the disclosure to a subject (e.g., a human subject) in need thereof, wherein the administering treats the disorder. In any of the aspects or embodiments of the disclosure, the disorder is a synucleinopathy. In various embodiments, the synucleinopathy is Lewy Body Dementia, Parkinson's disease, Gaucher's disease, or a combination thereof. In further embodiments, the Gaucher's disease is Type II or Type III. An “effective amount” of the ProSNA is an amount sufficient to, for example, treat, ameliorate, and/or prevent the disorder.

A ProSNA of the disclosure can be administered via any suitable route, for example and without limitation parenteral administration, intravenous, intraarterial, intraperitoneal, intranasal, intrathecal, and/or subcutaneous administration. A combination of different routes of administration, separately or at the same time, is also contemplated by the disclosure.

### Therapeutic Agents

In some embodiments, the ProSNAs provided herein further comprise a therapeutic agent, or a plurality thereof. In some embodiments, the therapeutic agent is associated with the protein core of the ProSNA. It is understood that the disclosure provides ProSNAs wherein one or more therapeutic agents are covalently and/or non-covalently associated with oligonucleotides in the shell of oligonucleotides that is attached to the exterior of the protein core of the ProSNA. It will also be understood that non-covalent associations include hybridization, protein binding, and/or hydrophobic interactions. In some embodiments, a therapeutic agent is administered separately from a ProSNA of the disclosure. Thus, in some embodiments, a therapeutic agent is administered before, after, or concurrently with a ProSNA of the disclosure to treat a disorder.

Therapeutic agents contemplated by the disclosure include without limitation a protein (e.g., a therapeutic protein), a growth factor, a hormone, an interferon, an interleukin, an antibody or antibody fragment, a small molecule, a peptide, an antibiotic, an antifungal, an antiviral, a chemotherapeutic agent, or a combination thereof.

The term “small molecule,” as used herein, refers to a chemical compound or a drug, or any other low molecular weight organic compound, either natural or synthetic. By “low molecular weight” is meant compounds having a molecular weight of less than 1500 Daltons, typically between 100 and 700 Daltons.

### EXAMPLES

### Example 1

GCase* ProSNA-TfF were successfully synthesized through the functionalization of activated GCase with folate terminated Transferrin Aptamer DNA. Oligonucleotides (TfR ((T)rans (F)errin (R)eceptor) is 5′-NH2—(SEQ ID NO: 1)-DBCO-dT-3′, wherein SEQ ID NO: 1 is 5′-GCG TGG TAC CAC GCT TTT T-3′) were made using standard phosphoramidite chemistry protocols using universal CPG solid supports and phosphoramidites, as well as coupling reagents purchased from Glen Research. Synthesized strands were purified by HPLC and analyzed using MALDI-MS. ProSNA synthesis was performed based on literature precedence. GCase from a CHO-expression system was first dissolved in PBS and coincubated with either queitiapine, ambroxol, or Saposin-C. Next 350 equivalents of NHS-PEG4-azide crosslinker were added to the activated GCase*, and the reaction was shaken overnight at 25° C. Unconjugated linker was removed by ten rounds of centrifugation using a 30 kDa filter, and the number of azide modifications was assessed by MALDI-MS. Finally, 350 equivalents of folate terminated DBCO-dT DNA strands were mixed with G-Case*-azide and allowed to incubate for 72 h at 25° C. with shaking. Unreacted DNA strands were removed by successive rounds of centrifugation in a 100 kDa filter until the filtrate did not have a detectable absorbance at 260 nm. The number of DNA strands per protein was calculated based on UV-Vis spectroscopy. Characterization of successful covalent conjugation was assessed by SDS PAGE gel.

### Materials and Methods

### Oligonucleotide Synthesis: All Discussed Sequences and Folate-DNA Conjugation

Transferrin (TfR) Aptamer Synthesis: The TfR aptamer was synthesized on solid CPG supports and terminated a 3′ DBCO-dT-CE phosphoramidite (5′-Dimethoxytrityl-5-[(6-oxo-6-(dibenzo[b,f]azacyclooct-4-yn-1-yl)-capramido-N-hex-6-yl)-3-acrylimido]-2′-deoxyUridine,3′-[(2-cyanoethyl)-(N,N-diisopropyl)]-phosphoramidite). Aptamer synthesis followed standard phosphoramidite chemistry protocols using purchased phosphoramidites and coupling reagents on a Biolytic Oligo Synthesizer yielding a final aptamer sequence of 5′NH₂-(SEQ ID NO: 1)-DBCO-dT-3′ (DBCO-TfR-NH₂), wherein SEQ ID NO: 1 is 5′-GCG TGG TAC CAC GCT TTT T-3′. Unless otherwise stated, all materials were purchased from Glen Research.

TfR Aptamer Purification and Characterization: Synthesized strands were cleaved from their CPG supports and purified by high performance liquid chromatography (HPLC). Representative fractions were collected and characterized through matrix-assisted laser desorption/ionization-time of flight (MALDI-TOF) mass spectrometry. Further purification and characterization were done through ethyl acetate extrusion and ultraviolet-visible (UV-VIS) spectrometry respectively, yielding concentration and percent yield per synthesis. Characteristic molecular weight measurements of DBCO-TfR, DBCO-TfR-NH₂, and DBCO-TfF with either a phosphodiester (PO) or phosphorothioate (PS) backbone are described in Table 1 below. All sequences described have an extinction coefficient of 177900 L/(mole*cm).

TABLE 1
Molecular weight values for synthesized oligonucleotides and
conjugated oligonucleotide strands. All discussed sequences
have an extinction coefficient of 177900 L/(mole*cm).

| | | | MW | MW |
|---|---|---|---|---|
| | Back- | | Expected | Observed |
| Name | bone | Sequence (5′ to 3′) | [Da] | [Da] |
| DBCO- | PS | (SEQ ID NO: 1)- DBCO-dT | 6863 | 6866 |
| TfR | | | | |
| DBCO- | PO | (SEQ ID NO: 1)- DBCO-dT | 6559 | 6563 |
| TfR | | | | |
| DBCO- | PS | NH₂-(SEQ ID NO: 1)- | 7714 | TBD |
| TfR-NH₂ | | DBCO-dT | | |
| DBCO- | PO | NH₂-(SEQ ID NO: 1)- | 7018 | 6993 |
| TfR-NH₂ | | DBCO-dT | | |
| DBCO- | PS | Folate-(SEQ ID NO: 1)- | 8252 | TBD |
| TfF | | DBCO-dT | | |
| DBCO- | PO | Folate-(SEQ ID NO: 1)- | 7531 | 7465 |
| TfF | | DBCO-dT | | |

Folate-TfR Conjugation: Purified DBCO-TfR-NH₂ was reacted with Folate-PEG3-Azide. Samples were prepared in PBS pH 7.4 at room temperature. The reaction was allowed to proceed for 4 hours. The solution then underwent 3 rounds of spin filtration followed by fast protein liquid chromatography (FPLC) purification. During the reaction, the azide group on the folate reacted with the primary amine on the 5′ end of the DBCO-TfR oligonucleotide sequence. This reaction resulted in a covalent linkage between the folate and DNA producing DBCO-TfF (transferrin-folate) strands. DBCO-TfF conjugation was characterized by MALDI-TOF mass spectrometry. The expected molecular weight measurements can be seen in Table 1.

### Synthesis of GCase* ProSNA-TfF: Activation and Post-Modification

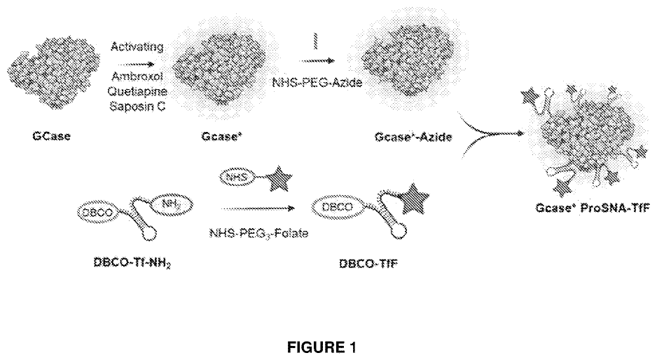

Glucosylceramidase (GCase) from a CHO-expression system was first dissolved in PBS and co-incubated with GCase activators quetiapine, ambroxol, or Saposin-C. Next 350 equivalents of NHS-PEG₄-azide crosslinker were added to activated GCase*, and the reaction was shaken overnight at 25° C. Unconjugated linker was removed through ten rounds of centrifugation using a 30 kDa filter, and the number of azide modifications was assessed by MALDI-TOF mass spectrometry. Finally, 350 equivalents of folate terminated DBCO-dT DNA strands were mixed with GCase*-azide and allowed to incubate for 72 h at 25° C. with shaking. Unreacted DNA strands were removed by successive rounds of centrifugation in a 50 kDa filter until the filtrate did not have a detectable absorbance at 260 nm. The number of DNA strands per protein was calculated based on UV-VIS spectroscopy. Characterization of successful covalent conjugation was assessed by SDS PAGE gel. A schematic representation of ProSNA TfR synthesis is described in FIG. 1.

### Characterization: DLS, CD, SDS

Characterization was carried out using several techniques including dynamic light scattering (DLS) for diameter characterization, circular dichroism (CD) for investigation into the secondary structure, folding, and binding properties of GCase, and sodium dodecyl-sulfate polyacrylamide gel electrophoresis (SDS-PAGE) for protein size and yield amount.

Dynamic Light Scattering: DLS measurements were taken using a Zetasizer NanoSZ using liposomal refractive index. Samples were prepared in PBS, and all measurements were conducted at 25° C. Final diameter values were taken as the number average of 4 measurements per sample.

Circular Dichroism: CD spectra were recorded in a 10 mm pathlength cuvette at 25° C. with a Jasco J-1700 spectropolarimeter equipped with a temperature controller at a concentration of 11.7 μM DNA or 300 nM GCase in 1×PBS buffer (pH 7.4). The instrument parameters to record the CD spectra were: 200-350 nm measurement range, 0.1 nm data pitch, 1 nm bandwidth, 50 nm/min scanning speed, and 5 accumulations. Calculated theoretical spectra of the ProSNA were obtained by adding the spectra of GCase-Activator to the spectra of the DNA.

Sodium Dodecyl-Sulfate Polyacrylamide Gel Electrophoresis: Protein samples were incubated at 80° C. for 5 min in 1× Laemmli Sample Buffer (Bio-Rad) and 355 mM 2-mercaptoethanol (Sigma-Aldrich) before loading. Samples were run on a 4-15% TGX mini-Protean pre-cast gradient gel (Bio-Rad) for 30 min at 200 V in 1× Tris/Glycine/SDS running buffer (Bio-Rad). Gels were stained using SimplyBlue SafeStain (Invitrogen) before imaging on a ChemiDoc™ MP Imaging System.

### Example 2

### Synthesis of Folate-Transferrin Bivalent Ligand

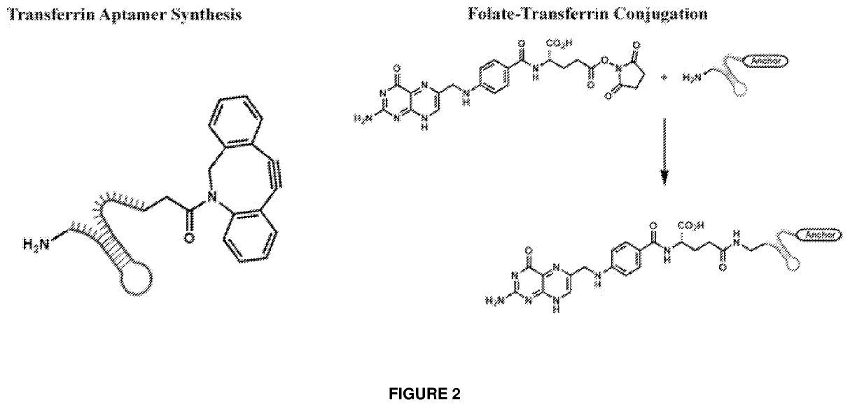

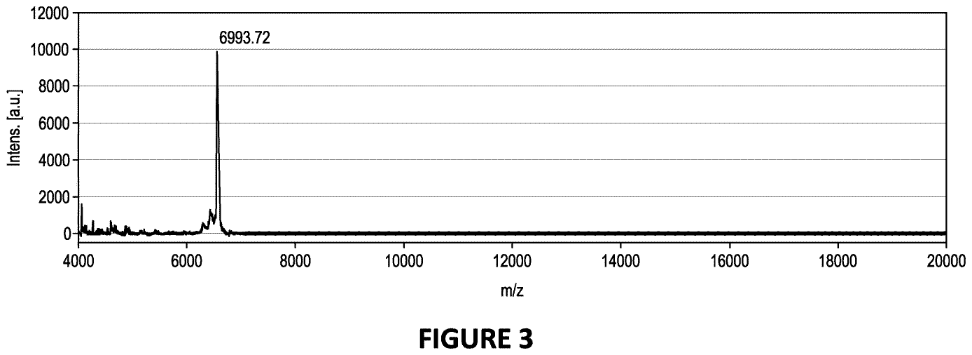

FIG. 2 depicts the synthetic scheme outlined for generation the proposed bivalent GCase ProSNA constructs. To synthesize the transferrin aptamer, standard phosphonamidite chemistry was used. The transferrin sequence (5′-Amino C6 dT-(SEQ ID NO: 1)-DBCO-dT-3′, wherein SEQ ID NO: 1 is 5′-GCG TGG TAC CAC GCT TTT T-3′) was made using a MerMade Oligonucleotide synthesizer in a 10 μmol scale on CPG solid supports and synthesized with a PO backbone. Once synthesized ligands were purified using high-performance liquid chromatography (HPLC). All oligonucleotides were purified on a 10 μm C4 column, using a gradient of 0-70% acetonitrile over 40 minutes. MALDI-TOF mass spectrometry was used to measure the molecular weight of collected fractions. Finally, the DMT group, used during synthesis to prevent polymerization of the nucleoside during functionalization of the support beads, was removed using three rounds of ethyl acetate extrusion. Successful ligands were verified by MALDI-TOF and compared to a calculated MW of 7017 Da. A representative MALDI spectra of successful ligand synthesis is shown in FIG. 3 where the observed MW was 6993.72 Da. Concentration of purified DNA was measured using ultraviolet visible spectroscopy (UV-VIS) where the optical density was measured at 260 nanometers. The concentration of working aptamer for all reactions discussed in this Example was 9.9 nmol/μL.

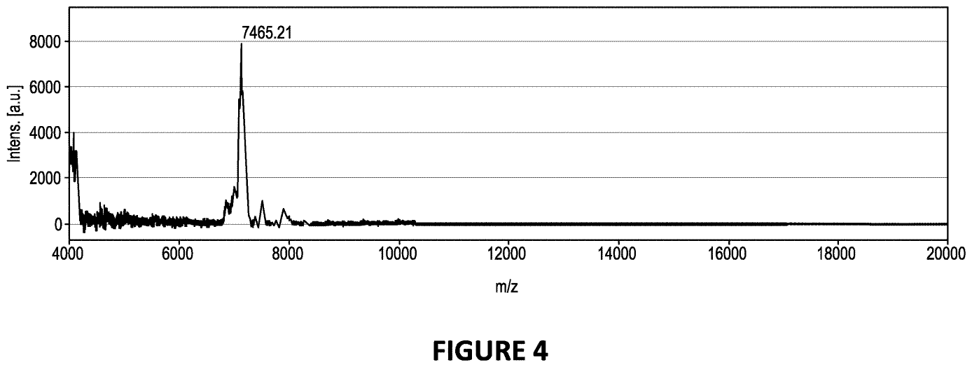

Once the transferrin aptamer was synthesized with the appropriate chemical handles, 500 nmol were reacted overnight with 50 equivalents of Folic Acid-NHS ester (Toronto Research Chemicals) prepared at 50 mg/mL in DMSO, in a 0.1M sodium carbonate buffer solution. Successful reactions were mixed 1:1 with 2,6-Dihydroxyacetophenone (DHAP), characterized by MALDI-TOF MS, and compared against a calculated MW of 7531 Da. A representative spectrum is shown in FIG. 4 and demonstrated a MW of 7465.21 Da. At this step, the NHS group on the folate reacts with the primary amine on the 5′ end of the aptamer sequence, forming a covalent bond and generating the bivalent DNA ligand.

### GCase-ProSNA Synthesis

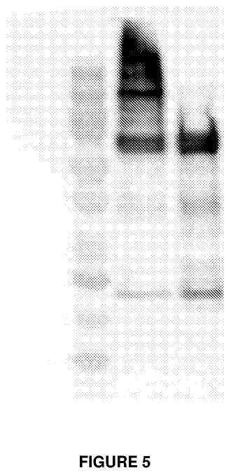

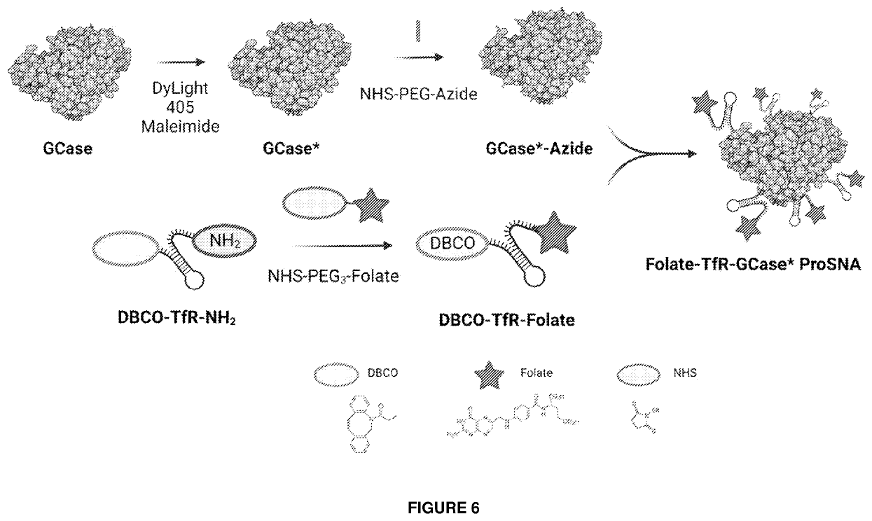

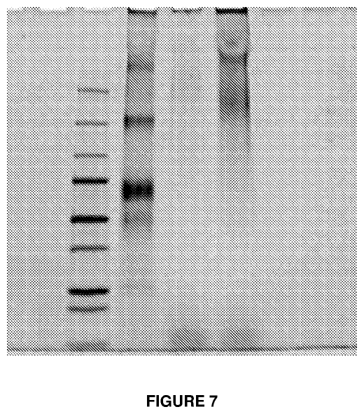

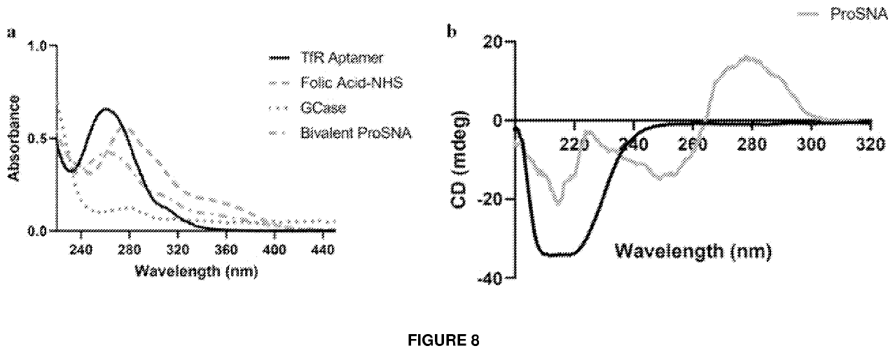

GCase from a CHO expression system (purchased from GenScript) was analyzed by western blot (FIG. 5) to confirm protein expression. Samples (stock concentration of 1.9 mg/mL) were incubated with 10% betamercaptoethanol to reduce cysteine-based dimer and trimer formation and run on a 4-12% Bis-Tris gel at 100V for 50 minutes. The gel was transferred using an iBlot dry transfer system (ThermoFisher) and stained with rabbit anti GCase (Sigma) primary antibody diluted 1:1000 in blocking buffer overnight at 4 degrees Celsius. The following day the gel was rinsed and stained with IR Dye 800CW goat anti rabbit (LI-COR) secondary antibody diluted 1:2000 in blocking buffer for 1 hour at room temperature. What is apparent from the gel is that the presence of betamercaptoethanol was able to reduce the cysteine-based dimer and trimer formation that was apparent in the untreated lane. Furthermore, the western blot ensured that GCase was properly expressed. Having confirmed the presence of GCase, the ProSNA could then be synthesized. An exemplary schematic of this process is seen in FIG. 6. Briefly, 40 nmol of GCase (1.9 mg/mL) was reacted with 20 equivalents of DyLight405-maleimide (ThermoFisher), prepared at 1 mg/mL in deionized water, overnight at room temperature. Unconjugated dye was removed through three rounds of centrifugation using a 30 kDa MWCO filter. To the purified sample, 500 equivalents of NHS-PEG₄-azide crosslinker, prepared at 2 mg/ml in deionized water, were added and the reaction was left to shake overnight at room temperature. The unconjugated crosslinker was removed through rounds of centrifugation using a 30 kDa spin filter. Finally, 50 equivalents of the DBCO terminated bivalent ligand, at 9.9 nmol/μL in deionized water, were introduced and the reaction was allowed to continue for 48 hours at room temperature. Unreacted DNA ligand was removed as before with centrifugation. Construct concentrations were calculated using Pierce BCA Protein Assay Kit (ThermoFisher) and varied based on the specific reaction. Reactions were characterized using several techniques including SDS-PAGE, UV-VIS, circular dichroism and MALDI-TOF mass spectrometry. To characterize by SDS-PAGE, samples were incubated with 2× loading dye for 5 minutes at 85 degrees Celsius. They were then run in a 4-15% SDS-PAGE gel at 200V for 34 minutes prior to being stained with Simply Blue. In the SDS-PAGE gel (FIG. 7) the unmodified protein was compared to the bivalent ProSNA. The first lane shows GCase with the expected MW of approximately 60 kDa present. Higher bands indicative of dimer and trimer formation were also seen. In the next lane a significant shift in electrophoretic mobility of the constructs was seen, indicative of successful functionalization. The new MW of the construct ranges from 170 kDa to 200 kDa which correlates to roughly 14-18 bivalent ligands functionalized to the surface and a loading density of 8.2-10.54 pmol/cm² respectively. The ProSNA were also characterized by UV-VIS spectroscopy in PBS (FIG. 8a). As evidenced in the spectra, when compared to the native protein alone, there is an increase in intensity at 260 nm correlating to the presence of the DNA aptamer. Furthermore, there is a broadening in the spectra at 280 and 305 nm which aligns with reported spectra data for Folic-Acid-NHS.

Additionally, circular dichroism was used to investigate the secondary structure of the bivalent ProSNA (FIG. 8b). When the protein is unmodified there is a decrease in ellipticity at 220 nm which is indicative of alpha helical structure. However, when the protein was functionalized with the bivalent ligand, the change was seen. Notably at 220 nm an increase in ellipticity was seen, which is indicative of DNA. These characterization methods confirmed that the bivalent GCase ProSNAs were successfully generated.

Further characterization of the constructs was carried out using MALDI-TOF mass spectrometry. The calculated MWs for the different constructs are outlined in Table 2, where the unmodified protein is GCase without any fluorescent tags or other modifications, univalent ProSNA is the GCase protein functionalized with only the transferrin aptamer, and the bivalent ProSNA is the GCase protein functionalized with the bivalent ligand characterized in FIG. 4. The expected MWs were calculated based on the assumption that 18 DNA ligands had been functionalized to represent total functionalization of surface lysine residues. MALDI reactions were carried out using different matrices including Sinapinic acid. Briefly, reaction samples were combined with four equivalents of Sinapinic acid. Solutions were then vortexed briefly and plated in 2 μL volumes, and measured in negative ion mode. The observed MW for GCase using this method is highlighted in Table 2. Final characterization of the ProSNA constructs may be performed in the same manner and serve to confirm the results discussed above.

TABLE 2
Comparison of calculated versus observed MW of unmodified
GCase protein, univalent ProSNA, and bivalent ProSNA.

| | Calculated | Observed |
|---|---|---|
| | MW (kDa) | MW (kDa) |
| Unmodified GCase Protein | 59.7 | 62.2 |
| Univalent ProSNA | 185.6 | TBD |
| Bivalent ProSNA | 194.1 | TBD |

### GCase Activity Measurements

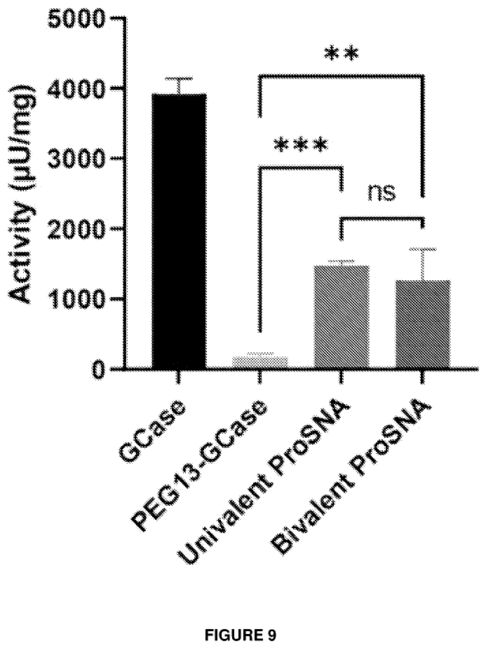

Following the synthesis process, a comprehensive investigation into the impact of synthesis on protein activity was conducted. To assess this, we employed a fluorometric GCase activity assay kit (Abcam), using a synthetic substrate-4-Methylumbelliferyl β-D-glucopyranoside. Upon interaction with active GCase, this substrate releases a fluorophore detectable using a plate reader at Ex/Em=360/445 nm. The focus extended to evaluating the effects of PEGylation on GCase, along with modifications involving univalent (sole presence of the transferrin aptamer) or bivalent (co-presence of both transferrin and folate) DNA ligands. By using the protocol outlined in this assay, the aim was to understand and analyze the variations in protein activity resulting from these modifications. Briefly, GCase constructs (all at 0.24 mg/mL) were reacted with GCase substrate for 30 minutes at 37° C. They were then measured for fluorescence intensity at Ex/Em=360/445. The results of this study are shown in FIG. 9. FIG. 9 indicates that while functionalization does impact protein activity there is no significant difference between the univalent and bivalent variations. Furthermore, there is a significant increase in activity when comparing the PEGylated protein to both ProSNA constructs. This was a key finding as PEGylation is currently one of the most used methods to improve protein delivery.

Activity is investigated via the co-incubation of ProSNA constructs with either ambroxol or quetiapine (a small molecule activator that has been shown to bind to GCase with high affinity thus increasing its activity). Such studies help determine whether the activity that is lost during functionalization can be recovered for maximal therapeutic effect. These studies are expected to show an elevation in enzyme activity through the co-assembly of small activators into the GCase ProSNA construct.

### Addition of Quetiapine Enhances Activity of GCase Protein

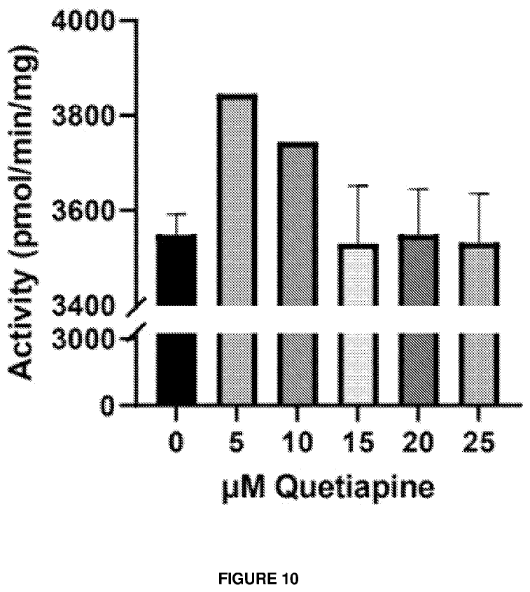

The purpose behind enzyme replacement therapy (ERT) is to replace protein that is either absent or dysregulated. Therefore, to achieve maximal therapeutic effect the protein being delivered needs to be in its most active form. As was demonstrated herein (FIG. 9), functionalization of the GCase protein with the bivalent DNA ligands led to a reduction of overall activity, with no significant difference between the uni- and bivalent constructs. To counteract this reduced activity, the native GCase protein was incubated with different concentrations (0, 5, 10, 15, 20, and 25 μM) of quetiapine keeping the concentration of GCase constant (1.9 mg/mL). Quetiapine is a small molecule that has been demonstrated to bind with high affinity (K_{d}=5 μM) to the GCase enzyme and has also demonstrated an ability to reduce the presence of α-synuclein protein aggregates which is the hallmark for Parkinson's Disease. This assay was completed using the same fluorometric kit described above, with quetiapine being introduced 30 minutes prior to the GCase substrate. The results of this study are shown in FIG. 10. Interestingly, it was found that 5 and 10 μM of quetiapine were sufficient to increase the activity of the native protein while the higher concentrations were not.

Next, the assay is repeated with generated GCase ProSNA constructs to investigate whether some of the activity lost during activation can be recovered. Furthermore, a second small molecule activator of GCase (Ambroxol—K_{d} of 182 μM at pH 7) is introduced to compare the activation capabilities. It is expected that the inclusion of the small molecules quetiapine and ambroxol will contribute to the partial recovery of GCase activity that may have been lost due to oligonucleotide functionalization.

### Flow Uptake, Models of Transcytosis, and IVIS Biodistribution

The GCase ProSNA constructs are characterized for therapeutic viability, including monitoring their uptake into different neuronal cell types at different time points, investigating their transcytotic potential across an in vitro model of the blood brain barrier (BBB), as well as determining their biodistribution when administered intravenously (retroorbital or tail vein injection) or intrathecally in vivo.

The ability of the GCase ProSNA constructs to be taken up into cells will be assessed. To investigate their uptake potential, flow cytometry is used as a method for determining the percent uptake of the constructs compared to control groups (unmodified GCase protein, linear bivalent DNA ligand, GCase-Transferrin aptamer, and GCase-Folic Acid). Constructs are synthesized as described previously, including the fluorescent tagging the GCase protein core as well as including a Cyanine3 dye into the aptamer sequence (Folate-SEQ ID NO: 1-Cy3-DBCO dT). Once prepared, the constructs are used to treat different mono-populations of different neuronal cell types, including neurons (dopaminergic), astrocytes, and microglia, to evaluate their uptake ability. In all cases, a live dead stain is used to determine percent uptake in viable populations. Furthermore, uptake across different time points is assessed to determine the rates at which the constructs are endocytosed and processed by the different cell types. It is expected that the presence of the multivalent DNA ligand functionalized to the GCase protein will enhance its uptake into all cell types when compared to highlighted control groups. Additionally, the rates of uptake are expected to be highest at shorter time points as the multivalent ligands enhance cellular recognition and subsequent cellular uptake.

The ability of the construct to pass the BBB is also assessed. This physiological defense prohibits the passing of any foreign substance including therapeutic agents. However, the GCase ProSNA is designed to overcome this challenge with the inclusion of the transferrin aptamer and folate groups that both serve as BBB targeting agents. This will enhance receptor mediated transcytosis thus increasing the percentage of ProSNA constructs that will make it to the brain space. To investigate this an in vitro transwell model of transcytosis is used. Human endothelial cells are cultured alone or in concert with microglial and astrocyte cells on a transwell membrane. Once confluent, the constructs are added to the apical side of the insert and their transcytosis through the cell monolayer to the basolateral side of the insert is quantified by fluorescence. It is expected that the presence of the multivalent DNA ligand on the GCase ProSNA constructs will allow for a higher rate of transcytosis when compared to control groups.

A current limitation of novel neurological therapeutics is non-specific biodistribution upon delivery. This decreases the concentration of therapeutic that actively reach the intended organ and conversely increases the concentration of therapeutic needed to be delivered to achieve a therapeutic effect. The biodistribution patterns of the GCase ProSNA is investigated using In Vivo Imaging System (IVIS). Fluorescently labeled constructs are administered to mice by different routes of injection including intravenously by way of retroorbital or tail vein injection, as well as intrathecally. At set time points the mice are imaged using IVIS to track the biodistribution patterns. It is expected that the ProSNA constructs will localize to the brain space in higher concentrations when compared to control groups due to the inclusion of the multivalent DNA ligand.

What is claimed is:

1. A protein-core spherical nucleic acid (ProSNA) comprising:
(a) a protein core that comprises a glucosylceramidase (GCase); and
(b) a shell of oligonucleotides attached to the protein core.

2. The ProSNA of claim 1, wherein the GCase is an activated GCase.

3. The ProSNA of claim 1, wherein the GCase is a non-activated GCase.

4. The ProSNA of any one of claims 1-3, wherein one or more oligonucleotides in the shell of oligonucleotides comprises a bivalent ligand.

5. The ProSNA of claim 4, wherein one or more oligonucleotides in the shell of oligonucleotides is a folate modified transferrin aptamer.

6. The ProSNA of any one of claims 1-5, wherein each oligonucleotide in the shell of oligonucleotides is a folate modified transferrin aptamer.

7. The ProSNA of claim 5 or claim 6, wherein the folate-modified transferrin aptamer comprises folic acid in the terminal position of the folate-modified transferrin aptamer.

8. The ProSNA of any one of claim 1, 2, 3, 4, 5, or 7, wherein the shell of oligonucleotides comprises one or more additional oligonucleotides.

9. The ProSNA of any one of claims 1-8, wherein one or more oligonucleotides in the shell of oligonucleotides is modified on its 5′ end and/or 3′ end with dibenzocyclooctyl (DBCO).

10. The ProSNA of any one of claims 1-9, wherein the shell of oligonucleotides comprises single-stranded DNA, double-stranded DNA, single-stranded RNA, double-stranded RNA, or a combination thereof.

11. The ProSNA of any one of claims 1-10, wherein each oligonucleotide in the shell of oligonucleotides is covalently attached to the protein core.

12. The ProSNA of claim 11, wherein each oligonucleotide in the shell of oligonucleotides is attached to the protein core through a linker.

13. The ProSNA of claim 12, wherein the linker is a cleavable linker or a non-cleavable linker.

14. The ProSNA of claim 12 or claim 13, wherein the linker is SPDP succinimidyl 3-(2-pyridyldithio) propionate.

15. The ProSNA of claim 14, wherein at least one oligonucleotide in the shell of oligonucleotides is attached to the protein core in the following configuration:
protein core—NH—C(O)—(CH₂)₅—NH—C(O)—C₂—S—S-oligonucleotide-NH-PEG₃-Folate.

16. The ProSNA of claim 12, wherein the linker is NHS-PEG_{(X)}-Azide, wherein X is 3 or 4.

17. The ProSNA of any one of claims 1-16, wherein at least one oligonucleotide in the shell of oligonucleotides is attached to the protein core in the following configuration:
protein core—NH—(CO)-PEG_{X}-Triazole-oligonucleotide-NH—C(O)-PEG₃-Folate.

18. A composition comprising a plurality of the protein-core spherical nucleic acids (ProSNAs) of any one of claims 1-17.

19. A method of treating, ameliorating, and/or preventing a synucleinopathy in a subject comprising administering to the subject an effective amount of (i) the ProSNA of any one of claims 1-17, (ii) the composition of claim 18, or (iii) a combination thereof.

20. The method of claim 19, wherein the synucleinopathy is Lewy Body Dementia, Parkinson's disease, Gaucher's disease, or a combination thereof.

21. The method of claim 20, wherein the Gaucher's disease is Type II or Type III.

22. A method of delivering a glucosylceramidase (GCase) to a cell comprising contacting the cell with the ProSNA of any one of claims 1-17.

23. A method of delivering a glucosylceramidase (GCase) to a cell comprising contacting the cell with the composition of claim 18.
